(19) United States
(12) Patent Application Publication
(10) Pub. No.: US 2018/0180555 A1
(43) Pub. Date: Jun. 28, 2018

(54) PROCESS FOR MONITORING THE CATALYTIC ACTIVITY OF AN IONIC LIQUID

(71) Applicants: SHELL OIL COMPANY, HOUSTON, TX (US); CHINA UNIVERSITY OF PETROLEUM, Beijing (CN)

(72) Inventors: Rui ZHANG, Beijing (CN); Zhichang LIU, Beijing (CN); Xuan ZHANG, Beijing (CN); Xianghai MENG, Beijing (CN); Haiyan LIU, Beijing (CN); Chunming XU, Beijing (CN); Peter Anton August KLUSENER, Amsterdam (NL)

(21) Appl. No.: 15/736,354

(22) PCT Filed: Jun. 16, 2016
(86) PCT No.: PCT/EP2016/063837

§ 371 Date: Dec. 14, 2017

(30) Foreign Application Priority Data
Jun. 18, 2015 (CN) PCT/CN2015/081770

(51) Int. Cl. G01N 21/79; G01N 21/35; B01J 31/02; C07C 2/68

(52) U.S. Cl. CPC G01N 21/79; G01N 21/35; C07C 2531/02; C07C 2/68; G01N 2021/3595; B01J 31/0279

(57) ABSTRACT

The present invention relates to a process for monitoring the catalytic activity of an ionic liquid. In step (a), providing an acidic ionic liquid; (b) providing an organic compound; (c) adding at least a portion of the organic compound to at least a portion of the ionic liquid; (d) recording an infrared spectrum of a mixture from step (c) to obtain at least one absorption peak. In step (e), repeating steps (c) and (d) until at least one absorption peak reaches a maximum value or a minimum value. In step (f), determining at the maximum value or minimum value of step (e): the total amount of the organic compound or the total amount of the ionic liquid added. In step (g), calculating the catalytic activity of the ionic liquid based on: the total amount of the organic compound or the total amount of ionic liquid, as determined in step (f).

### FIELD OF THE INVENTION

The present invention provides a process for monitoring the catalytic activity and a process for preparing an alkylate using an ionic liquid of which the catalytic activity of the ionic liquid is determined using said monitoring process.

### BACKGROUND OF THE INVENTION

Acidic ionic liquids (ILs), such as chloroalumininates, are successfully being used as environmentally friendly catalysts for the alkylation of 2-butene with isobutane or of benzene with an alphaolefin or an alkyl halide. The control of the catalytic activity and the regeneration of these ILs are important features for the industrial application. Catalytic activity is related to the acidity of these acidic ionic liquids. Therefore, interest in methods for monitoring the acidity of ionic liquids to enable and improve the control of the active species in ionic liquids has increased.

US2011/0184219 discloses a process to determine the ionic liquid catalyst deactivation by hydrolyzing a sample of ionic liquid catalyst, followed by titrating the hydrolyzed sample with a basic reagent to determine a volume of the basic reagent necessary to neutralize a Lewis acid species of the ionic liquid catalyst. The acid content from the sample in US2011/0184219 is then calculated from the volume of the used basic reagent.

WO2012/158259 discloses a method for monitoring an ionic liquid by contacting an infrared (IR) transmissive medium with the ionic liquid, followed by recording an IR spectrum of the ionic liquid, from which spectrum at least one chemical characteristic of the ionic liquid is quantified.

A problem of the processes disclosed in US2011/0184219 and WO2012/158259 is that said processes do not quantitatively characterize the activity of ionic liquids. In this way the level of catalytic activity of the ionic liquids cannot be monitored and consequently by lack of quantitative information on the extent of deactivation, the control of the regeneration in the process of continuous alkylation is difficult.

It is an object of the invention to provide a quantitative characterization method for the catalytic activity of acidic ionic liquids.

It is a further object of the present invention to monitor the catalytic activity level of acidic ionic liquids and to define the activity level of the ionic liquid at which corrective actions are necessary to maintain catalyst activity and ensuring a continuous alkylation process.

One of the above or other objects may be achieved according to the present invention to provide a process for monitoring the catalytic activity of an ionic liquid, comprising the steps of:

(a) providing an acidic ionic liquid;
(b) providing an organic compound which contains a nitrogen group, oxygen group and/or sulphur group;
(c) adding a portion of the organic compound to a sample of the ionic liquid or adding a portion of the ionic liquid to a sample of the organic compound;
(d) recording an infrared spectrum of a mixture as obtained in step (c) to obtain at least one absorption peak;
(e) repeating steps (c) and (d) until at least one absorption peak obtained in step (d) reaches a maximum value or a minimum value;
(f) determining at the maximum value or minimum value of the absorption peak of step (e): the total amount of the organic compound added in portions to the sample of the ionic liquid or determining the total amount of the ionic liquid added in portions to the sample of organic compound;
(g) calculating the catalytic activity of the ionic liquid on the basis of: the total amount of the organic compound added in portions as determined in step (f) or the total amount of ionic liquid added in portions as determined in step (f).

It has now surprisingly been found according to the present invention that the catalytic activity of ionic liquids can be quantitatively characterized.

It is known that the catalytic activity of acidic chloroaluminate ionic liquids originates from the Lewis acid in chloroaluminate ionic liquids. The relationship between the catalytic activity of chloroaluminate ionic liquids and the Lewis acid Al₂Cl₇⁻ in the chloroaluminate ionic liquids is described in for instance J. Cui, J. de With, P. A. A. Klusener, X. H. Su, X. H. Meng, R. Zhang, Z. C. Liu, C. M. Xu and H. Y. Liu, “Identification of acidic species in chloroaluminate ionic liquid catalysts”, J. Catal., 320 (2014) 26.

By using in-situ infrared-complexometric titration the Al₂Cl₇⁻ active component in the chloroaluminate ionic liquids during continuous alkylation can be quantitatively characterized.

In this way, the catalytic activity of the ionic liquid can be monitored with complexometric titration, by using infrared spectroscopy, preferably by using in-situ infrared spectroscopy.

Another advantage of the present invention is that by monitoring the catalytic activity of an ionic liquid, it can be determined at which activity the alkylation activity is too low for total conversion of the olefin. Therewith, the level of required activity of the catalyst (ionic liquid) as monitored by the method described above can be defined and used to control the catalyst activity in the process.

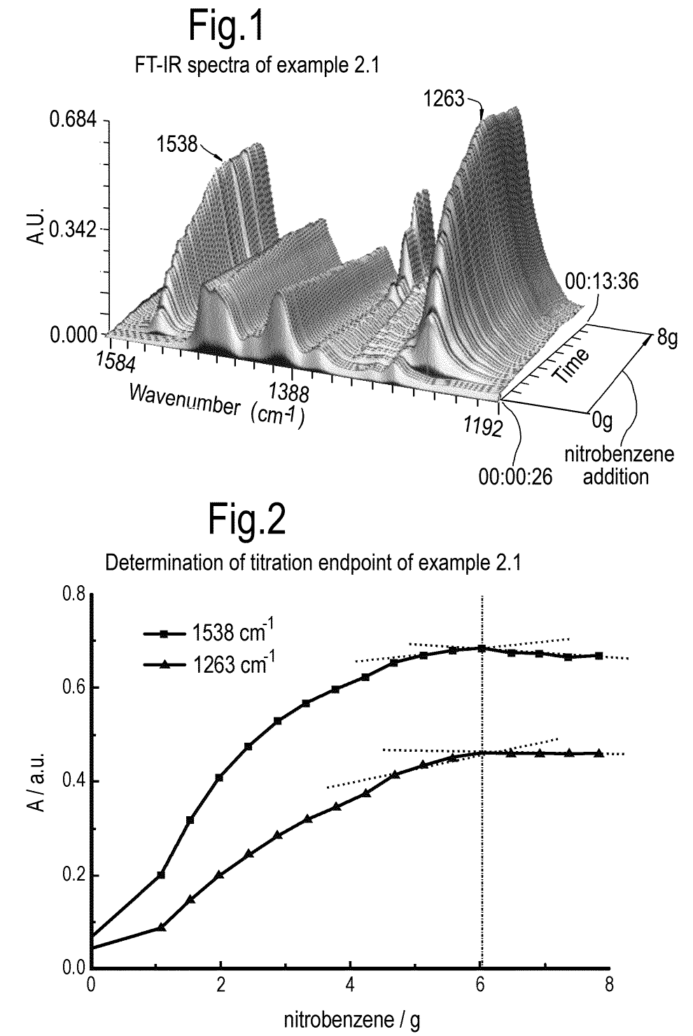

In FIGS. 1, 5, 7, 10, 13, and 15 infrared-spectra of titration of IL with organic compounds are shown.

In FIGS. 2, 6, 8, 9, 11, 12, and 14, the determination of the titration endpoints of the titration of IL with organic compounds are shown.

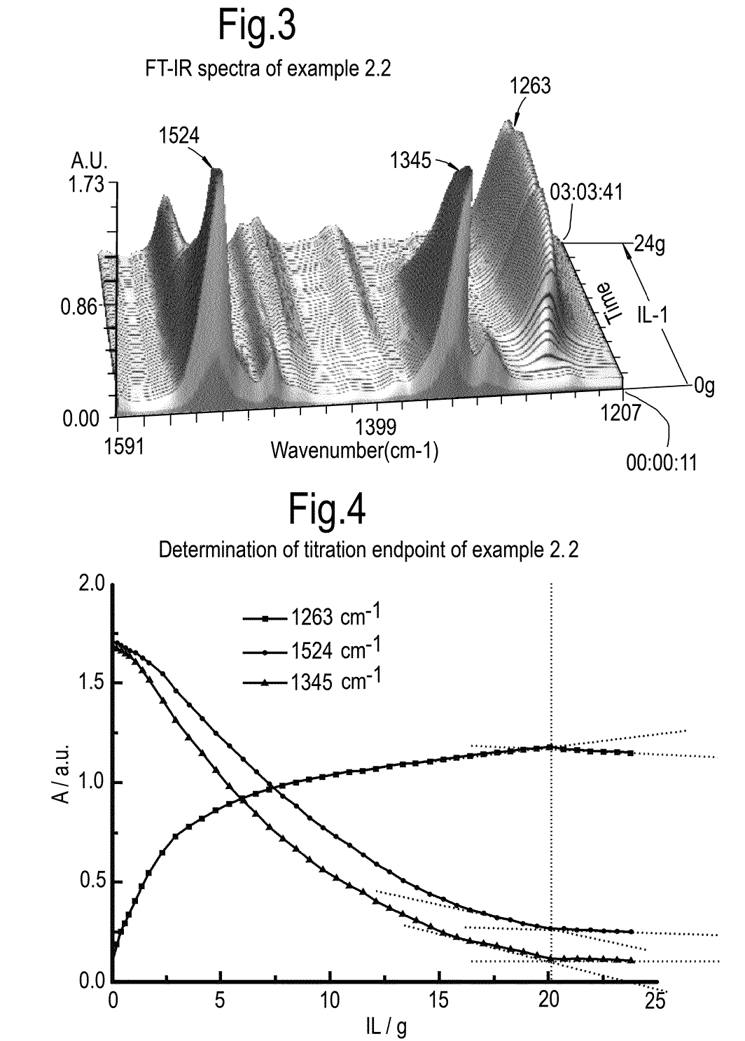

In FIG. 3, an infrared spectrum of titration of an organic compound with IL is shown.

In FIG. 4 the determination of the titration endpoints of the titration of an organic compound with IL is shown.

In step (a) of the process according to the present invention an acidic ionic liquid is provided. Processes to prepare ionic liquids are known in the art and are therefore not discussed here in detail. Preparation of acidic ionic liquids is for example described in U.S. Pat. No. 7,285,698, WO2011/015639 and WO2015/028514.

Preferably, the acidic ionic liquid is a chloroaluminate ionic liquid. The preparation of an acidic chloroaluminate ionic liquid has been described in e.g. WO2015/028514.

In step (b) of the process according to the present invention an organic compound which contains a nitrogen group, oxygen group and/or sulphur group is provided.

Preferably, the organic group which contains a nitrogen group, oxygen group and/or sulphur group is selected from the group consisting of alcohols, ketones, ethers, tetrahydrofurans, aldehydes, mercaptans, sulphur ethers, thiophenes, pyridines, nitro-aromates and derivatives thereof.

More preferably, the organic group which contains a nitrogen group, oxygen group and/or sulphur group is selected from the group consisting of ethanol, acetone, diethyl ether, tetrahydrofuran, nitrobenzene, meta-methyl nitrobenzene, pyridine, and 2,6-dimethyl pyridine.

Most preferred organic group is nitrobenzene, acetone, tetrahydrofuran, ethanol, or diethyl ether.

In step (c) of the process according to the present invention a portion of the organic compound is added to a sample of the ionic liquid or a portion of the ionic liquid is added to a sample of the organic compound.

In a first embodiment in step (c) of the process according to the present invention a portion of the organic compound is added to a sample of the ionic liquid to obtain a mixture.

In a second embodiment in step (c) of the process according to the present invention a portion of the ionic liquid is added to a sample of the organic compound to obtain a mixture.

Preferably, a sample of the ionic liquid is titrated with a portion of the organic compound or a sample of the organic compound is titrated with a portion of the ionic liquid. More preferably, the titration is complexometric titration. Titration, and in specific complexometric titration, is a technique known in the art and therefore not described here in detail.

Complexometric titration techniques are for example described in G. Schwarzenbach and H. A. Flasch, “Complexometric titrations”, 2^{nd} Ed., Methuen (1969).

This principle of the titration in this embodiments is related to monitoring the formation of a product between the organic compound and the acidic species in the ionic liquid and/or in case of adding ionic liquid for the organic compound also to the monitoring the disappearance of the organic compound. The monitoring in step (d) can be performed using spectroscopic techniques.

Suitably, the organic compound or the ionic liquid is used as a mixture using a solvent as diluent, preferred solvent is dichloromethane. Dichloromethane is the preferred solvent since said solvent does not react with the ionic liquid.

Preferably, the ionic liquid is used as a mixture using a solvent as diluent.

By using a solvent for the ionic liquid the advantage is that it lowers the viscosity and makes the mixing with the organic compound faster. So it fastens the reaction between acidic sites of the ionic liquid and organic compound and makes the titration more accurate.

The volume ratio of the solvent to the ionic liquid or the organic compound is preferably 0.5 to 20.

In step (d) of the process according to the present invention an infrared spectrum of the mixture as obtained in step (c) is recorded to obtain at least one absorption peak. Preferably, the infrared spectrum is recorded with a Fourier Transform Infrared Spectrometer (FT-IR). The use of FT-IR for following titration is a method known in the art and therefore not described here in detail. FT-IR for following titration is for example described in D. Li, J. Sedman, D. L. Garcia-Gonzalez, and F. R. van de Voort, “Automated Acid Content Determination in Lubricants by FTIR Spectroscopy as an Alternative to Acid Number Determination”, Journal of ASTM International, Vol. 6, No. 6 (2009) Paper ID JAI102110.

Preferably, the infrared spectrum of steps (d) en (e) is recorded in situ during step (c), (d) and (e).

In the present invention by the term “in situ” is meant recording infrared spectra during titration.

The absorption peak in step (d) may result from the reaction product between the ionic liquid and the organic compound. In step (d) preferably one or more absorption peaks are obtained corresponding to one or more reaction products between the ionic liquid and the organic compound. Preferably, the absorption peak may result from the reaction product between the acidic chloroaluminate ionic liquid and the functional groups in the organic compounds containing nitrogen, oxygen and/or sulphur.

In alternative embodiments of this invention, in step (d) of the process according to the invention a Nuclear Magnetic Resonance (NMR) spectrum of a mixture as obtained in step (c) is recorded to obtain signals related to the reaction product of acidic ionic liquid and the organic compound and/or the disappearance of the organic compound. In other alternative embodiments of this invention other analytical techniques are used, such as ultra violet spectroscopy or colourimetry, that are sensitive to selectively monitor the formation of the reaction product of acidic ionic liquid and the organic compound and/or the disappearance of organic compound.

In the case that in step (c) a portion of the ionic liquid is added to a sample of the organic compound, the absorption peak in step (d) may result from the organic compound. Preferably, the absorption peak may result from the functional groups in the organic compounds containing nitrogen, oxygen or sulphur.

In step (e) of the process according to the present invention steps (c) and (d) are repeated until at least one absorption peak obtained in step (d) reaches a maximum value or a minimum value.

In the case that in step (c) a portion of the organic compound is added to a sample of the ionic liquid, at least one of the absorption peaks corresponding to one or more reaction products between the ionic liquid and the organic compound preferably reaches a maximum value in step (e).

In the case that in step (c) a portion of the ionic liquid is added to a sample of the organic compound, at least one absorption peak resulting from the organic compound reaches a minimum in step (e). As indicated above, the absorption peak may result from the functional groups in the organic compounds containing nitrogen, oxygen or sulphur.

In a first embodiment of the present invention, in step (c) a portion of the organic compound is added to a sample of ionic liquid to obtain a mixture. Preferably, of this mixture an infrared spectrum is recorded in step (d) to obtain a first absorption peak. In step (e) the first absorption peak corresponding to a first product between the ionic liquid and the organic compound reaches a maximum and at further repeating steps (c) en (d) a second absorption peak corresponding to a second product between the ionic liquid and organic compound reaches a maximum.

This second product may result from reaction between the first product and the functional groups in the organic compounds containing nitrogen, oxygen and/or sulphur. The first product may therefore be converted in the second product and may therefore disappear. Therefore, in step (e) at least one absorption peak corresponding to a product between the ionic liquid and the organic compound reaches a maximum and at further repeating steps (c) en (d) the same absorption peak reaches a minimum.

Preferably, depending on the type of organic compound used in step (c) in step (e) at least one absorption peak corresponding to a product between the ionic liquid and the organic compound reaches a maximum and at further repeating steps (c) en (d) the same absorption peak reaches a minimum. Typically, some organic compound result in a second absorption peak.

In step (f) of the process according to the present invention, at the maximum value or minimum value of the absorption peak of step (e) the total amount of the organic compound added in portions to the sample of the ionic liquid is determined or the total amount of the ionic liquid added in portions to the sample of organic compound is determined.

In the first embodiment as indicated above, by addition of the organic compound in portions to the sample of the ionic liquid in step (e) at least one absorption peak corresponding to a product between the ionic liquid and the organic compound reaches a maximum and at further repeating steps (c) en (d) the same absorption peak reaches a minimum.

Therefore, in step (f) of the first embodiment the total amounts of the organic compound added in portions to the sample of the ionic liquid is determined at which in step (e) one or more absorption peaks corresponding to a product between the ionic liquid and the organic compound reach a maximum or a minimum after first having reached a maximum.

In the second embodiment of the present invention, in step (c) a portion of the ionic liquid is added to a sample of the organic compound to obtain a mixture.

Typically, in the beginning of step (c) an infrared spectrum of only the organic compound is recorded because a reaction product between the ionic liquid and the organic compound may have not be formed.

Preferably, in step (d) at least one absorption peak is obtained corresponding to the organic compound. As more ionic liquid is added to a sample of organic compound a product may be obtained resulting from reaction between the acidic ionic liquid, preferably chloroaluminate ionic liquid, and the functional groups in the organic compounds containing nitrogen, oxygen and/or sulphur. The organic compound may therefore be converted in said product and may therefore disappear.

In the second embodiment as indicated above, by addition of the ionic liquid in portions to the sample of the organic compound, in step (e) a minimum is reached of the absorption peak corresponding to the organic compound and a maximum of the absorption peak corresponding to a product between the ionic liquid and the organic compound.

Therefore, in step (f) of the second embodiment the total amount of the ionic liquid added in portions to the sample of organic compound is determined at which in step (e) a minimum is reached of the absorption peak corresponding to the organic compound or a maximum of the absorption peak corresponding to the product between the ionic liquid and the organic compound.

In step (g) of the process according to the present invention the catalytic activity of the ionic liquid is calculated on the basis of: the total amount of the organic compound added in portions as determined in step (f) or the total amount of ionic liquid added in portions as determined in step (f).

The catalytic activity of the ionic liquid according to the present invention is defined as the ratio between the amount of organic compound and the amount of ionic liquid added at reaching the minimum or maximum as obtained in step (e).

In practice any unit for the ratio of amounts of organic compound and ionic liquid can be used to define an “activity index” as appropriate for the specific combination of organic compound and ionic liquid, such as: g indicator/100 g IL, mol indicator/mol IL, etc.

The catalytic activity may for instance be calculated with the formula as indicated below.

$${AI}_{IL } = \frac{100 \cdot {m}_{IN}}{{m}_{IL} \cdot {M}_{IN}}$$

In the formula, AI_{IL} is the “activity index” of the ionic liquid, m_{IN} is the mass of the organic compound usage at the titration end point or the mass of the sample of organic compound in case ionic liquid was added to the organic compound, M_{IN} is the molecular mass of the organic compound, and m_{IL} is the mass of the sample of the ionic liquid or the mass of the amount of ionic liquid added to the organic compound sample at the titration end point.

In the first embodiment, in step (g) the catalytic activity of the ionic liquid is determined by the ratio of the total amount of the organic compound added in portions as determined in step (f) and the amount of the sample of ionic liquid of step (c).

In the second embodiment, in step (g) the catalytic activity of the ionic liquid is determined by the ratio of the amount of the sample of organic compound of step (c) and the total amount of ionic liquid added in portions as determined in step (f).

In a further aspect the present invention provides a process to prepare an alkylate product, the process at least comprising the steps:

(aa) providing a hydrocarbon mixture comprising at least an isoparaffin or an aromatic hydrocarbon and an olefin;
(bb) subjecting the mixture of step (aa) to an alkylation reaction between the isoparaffin or the aromatic hydrocarbon and the olefin, wherein the hydrocarbon mixture is reacted with an ionic liquid to obtain an effluent comprising at least an alkylate product;
(cc) separating the effluent of step (bb), thereby obtaining a hydrocarbon-rich phase and an ionic liquid-rich phase;
(dd) fractionating the hydrocarbon-rich phase of step (cc), thereby obtaining at least the alkylate product and a isoparaffin-comprising stream or an aromatic hydrocarbon-comprising stream; and
(ee) recycling of the ionic liquid-rich phase of step (cc) to step (bb), wherein the catalytic activity of the ionic liquid of step (bb) and of the ionic liquid rich phase (cc) is determined with a process for monitoring the catalytic activity of an ionic liquid according to the present invention.

Process to prepare an alkylate product are known in the art and therefore not described here in detail. Process to prepare alkylate products comprising steps (aa) to (ee) are for example described in U.S. Pat. No. 7,285,698, WO2011/015639, in WO2015/028514 and US20100160703, but the processes disclosed in the prior art, such as U.S. Pat. No. 7,285,698, WO2011/015639, WO2015/028514 and in US20100160703 do not include determination of the catalytic activity of the ionic liquid of step (bb) and of the ionic liquid rich phase of step (cc) as determined with the process for monitoring the catalytic activity of an ionic liquid according the present invention

The invention is illustrated by the following non-limiting examples.

### EXAMPLE 1 PREPARATION OF IONIC LIQUID (IL)

### Example 1.1 Preparation of Ionic Liquid Et₃NHCl-2.0AlCl₃ (IL-1)

Et₃NHCl and AlCl₃ were obtained from Aladdin Industrial Inc.

137.7 g of Et₃NHCl (1 mol) was placed in a 500 mL flask under N₂ atmosphere. Subsequently, 133.3 g of AlCl₃ (1 mol) was added into the flask. A reaction started and the mixture was stirred while the temperature rose to 100° C. by the exothermic reaction. The mixture was heated as soon as the temperature started to drop and kept at 120° C. for at least 2 hours by external heating. Then another portion of 133.3 g of AlCl₃ (1 mol) was added into the flask. The temperature of the mixture rose to 150° C. The temperature of mixture was kept at 150° C. for at least 4 hours using external heating until a homogeneous liquid was obtained. The resulting liquid, being 404.3 g of ionic liquid IL-1, was allowed to cool down to room temperature.

### Example 1.2 Preparation of Ionic Liquid Et₃NHCl-1.8 AlCl₃ (IL-2)

The procedure of example 1.1 was repeated using in the second portion of AlCl₃ 106.7 g (0.8 mol) instead of 133.3 g of AlCl₃ (1.0 mol). 377.7 g of IL-2 was obtained.

### Example 1.3 Preparation of Ionic Liquid Et₃NHCl-1.6 AlCl₃ (IL-3)

The procedure of example 1.1 was repeated using in the second portion of AlCl₃ 80.0 g (0.6 mol) instead of 133.3 g of AlCl₃ (1.0 mol). 351 g of IL-3 was obtained.

### Example 1.4 Preparation of Ionic Liquid Et₃NHCl-1.4 AlCl₃ (IL-4)

The procedure of example 1.1 was repeated using in the second portion of AlCl₃ 53.3 g of AlCl₃ (0.4 mol) instead of 133.3 g of AlCl₃ (1.0 mol). 324.3 g of IL-4 was obtained.

### Example 1.5 Preparation of Ionic Liquid Et₃NHCl-1.2 AlCl₃ (IL-5)

The procedure of example 1.1 was repeated using in total 26.7 g of AlCl₃ (0.2 mol) instead of 133.3 g of AlCl₃ (1.0 mol). 297.7 g of IL-5 was obtained.

### Example 1.6 Preparation of Composite Ionic Liquid Et₃NHCl-1.8AlCl₃-0.2CuCl (IL-6)

Et₃NHCl, AlCl₃, and CuCl were obtained from Aladdin Industrial Inc.

137.7 g of Et₃NHCl (1 mol) was placed in a 500 mL flask under N₂ atmosphere. Subsequently, 133.3 g of AlCl₃ (1 mol) was added into the flask. A reaction started and the mixture was stirred while its temperature rose to 100° C. by the exothermic reaction. When the temperature of the mixture had decreased below 60° C. by slowly cooling, 19.8 g of CuCl (0.2 mol) was added to the mixture. The temperature of the mixture rose due the heat of reaction. The IL mixture was heated as soon as its temperature started to drop and the temperature of the mixture was kept at 120° C. for at least 2 hours by external heating. Then another portion of 106.7 g of AlCl₃ (0.8 mol) was added into the flask. The temperature of the mixture rose to 150° C. The temperature of mixture was kept at 150° C. for at least 4 hours using external heating until a homogeneous liquid was obtained. The resulting liquid, being 397.5 g of composite ionic liquid IL-6, was allowed to cool down to room temperature.

### Example 1.7 Preparation of Ionic Liquid Et₃NHCl-2.0AlBr₃ (IL-7)

Et₃NHCl and AlBr₃ were obtained from Aladdin Industrial Inc.

137.7 g of Et₃NHCl (1.0 mol) was placed in a 500 mL flask under N₂ atmosphere. Subsequently, 266.7 g of AlBr₃ (1.0 mol) was added into the flask. A reaction started and the mixture was stirred while the temperature rose to 100° C. by the exothermic reaction. The mixture was heated as soon as the temperature started to drop and kept at 120° C. for at least 2 hours by external heating. Then another portion of 266.7 g of AlBr₃ (1.0 mol) was added into the flask. The temperature of the mixture rose to 150° C. The temperature of mixture was kept at 150° C. for at least 4 hours using external heating until a homogeneous liquid was obtained. The resulting liquid, being 671.1 g of ionic liquid IL-7, was allowed to cool down to room temperature.

### EXAMPLE 2 DETERMINATION OF CATALYTIC ACTIVITY OF IL WITH INFRARED SPECTROSCOPY

### Example 2.1 Determination of Catalytic Activity of IL-1 with Infrared Spectroscopy by Titration of IL-1 with Nitrobenzene

IL-1 (20.012 g) was placed in a 50 mL flask under N₂ atmosphere and was stirred continuously during the titration. The titration was performed by addition of nitrobenzene (supplied by Aladdin Company) in portions while FT-IR spectra of the mixture were recorded in situ by an infrared detection apparatus at equal time intervals. FIG. 1 shows that during the titration absorption peaks at 1263 cm⁻¹ and 1538 cm⁻¹ appeared and the intensity of these two peaks increased gradually as nitrobenzene was added in portions. The intensity changes of these two peaks were tracked in-situ by the infrared apparatus and plotted against the amount of nitrobenzene added (FIG. 2). The titration end point was defined as the point whereby upon the further addition of nitrobenzene the intensities of the two peaks did not increase anymore. The nitrobenzene usage at the titration end point was 6.025 g. The catalytic activity defined as the “activity index” of IL-1 ionic liquid was 0.245 mol indicator/100 g of IL.

### Example 2.2 Determination of Catalytic Activity of IL-1 with Infrared Spectroscopy by Titration of Nitrobenzene with IL-1

Nitrobenzene (6.010 g, supplied by Aladdin Company) was placed in a 50 mL flask under N₂ atmosphere and was stirred continuously during the titration. The titration was performed by addition of IL-1 in portions while FT-IR spectra of the mixture were recorded in situ by an infrared detection apparatus at equal time intervals. FIG. 3 shows that during titration an absorption peak at 1263 cm⁻¹ appeared and gradually increased, while the peaks at 1524 cm⁻¹ and 1345 cm⁻¹ gradually decreased. The intensity changes of these three peaks were tracked by the infrared apparatus and plotted against the amount of IL-1 added (FIG. 4). The titration end point was defined as the point whereby upon the further addition of IL-1 the intensities of the peaks did not increase or decrease anymore. The IL-1 usage at the titration end point was 20.058 g. The catalytic activity defined as the “activity index” of IL-1 ionic liquid was 0.243 mol indicator/100 g of IL.

### Example 2.3 Determination of Catalytic Activity of IL-2 with Infrared Spectroscopy by Titration of IL-2 with Nitrobenzene

The procedure of example 2.1 was repeated for determining the catalytic activity of IL-2 (20.003 g) by titration with nitrobenzene. The nitrobenzene usage at the titration end point was 5.151 g. The “activity index” of IL-2 was 0.209 mol indicator/100 g of IL.

### Example 2.4 Determination of Catalytic Activity of IL-3 with Infrared Spectroscopy by Titration of IL-3 with Nitrobenzene

The procedure of example 2.1 was repeated for determining the catalytic activity of IL-3 (20.007 g) by titration with nitrobenzene. The nitrobenzene usage at the titration end point was 4.158 g. The “activity index” of IL-3 was 0.169 mol indicator/100 g of IL.

### Example 2.5 Determination of Catalytic Activity of IL-4 with Infrared Spectroscopy by Titration of IL-4 with Nitrobenzene

The procedure of example 2.1 was repeated for determining the catalytic activity of IL-4 (20.005 g) by titration with nitrobenzene. The nitrobenzene usage at the titration end point was 3.037 g. The “activity index” of IL-4 was 0.123 mol indicator/100 g of IL.

### Example 2.6 Determination of Catalytic Activity of IL-5 with Infrared Spectroscopy by Titration of IL-5 with Nitrobenzene

The procedure of example 2.1 was repeated for determining the catalytic activity of IL-5 (20.007 g) by titration with nitrobenzene. The nitrobenzene usage at the titration end point was 1.665 g. The “activity index” of IL-5 was 0.068 mol indicator/100 g of IL.

### Example 2.7 Determination of Catalytic Activity of IL-1 with Infrared Spectroscopy by Titration of IL-1 with Acetone

IL-1 (20.012) g was placed in a 50 mL flask under N₂ atmosphere and was stirred continuously during the titration. The titration was performed by addition of acetone (supplied by Aladdin Company) in portions while FT-IR spectra of the mixture were recorded in situ by an infrared detection apparatus at equal time intervals.

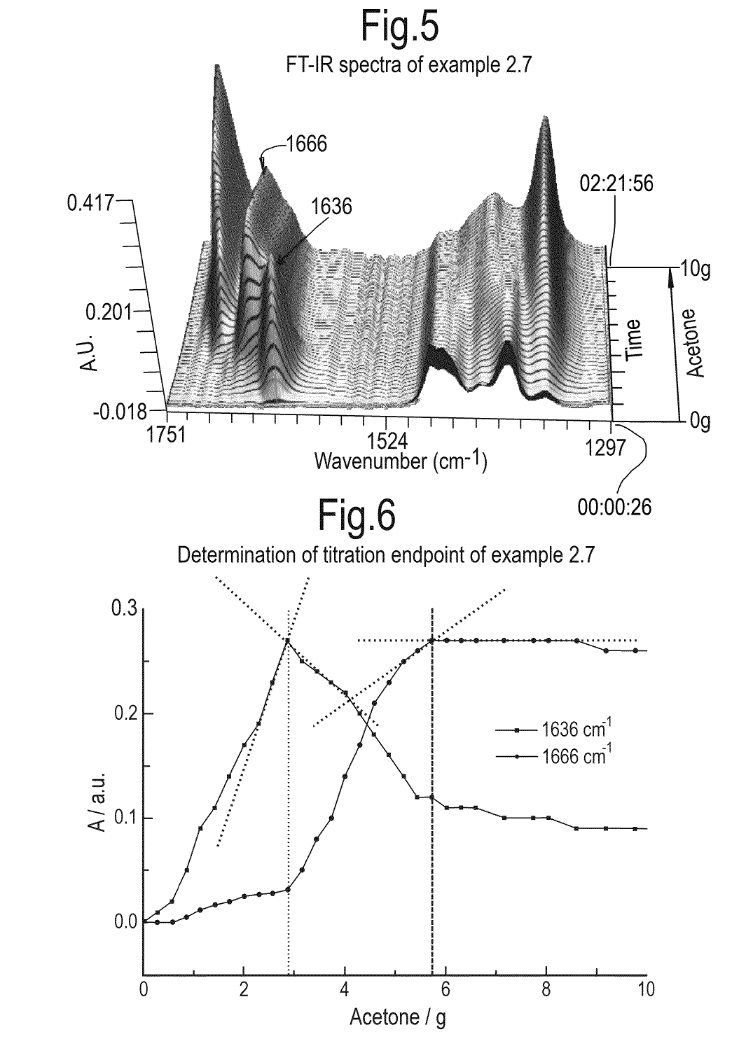

FIG. 5 shows that during titration initially an absorption peak at 1666 cm⁻¹ appeared and when it reached its maximum another peak at 1636 cm⁻¹ appeared, while acetone was added in portions. The intensity changes of these two peaks were tracked by the in-situ infrared apparatus and plotted against the amount of acetone added (FIG. 6). The titration end points were determined at the moment that the intensity of the 1636 cm⁻¹ peak reached its maximum and when the intensity of the 1666 cm⁻¹ was not increasing anymore upon the addition of acetone. The acetone usage was 2.861 g at the first titration end point and 5.7 g at the second titration end point. The second titration end point is related to the interaction of two molar equivalents of acetone with the catalyst; so the acetone usage at this second titration end point needs to be divided by 2, to be used in the calculation of the catalytic activity. The catalytic activity of IL-1 ionic liquid defined as “activity index” was 0.246 mol indicator/100 g of IL.

### Example 2.8 Determination of Catalytic Activity of IL-1 with Infrared Spectroscopy by Titration of IL-1 with Tetrahydrofuran (THF)

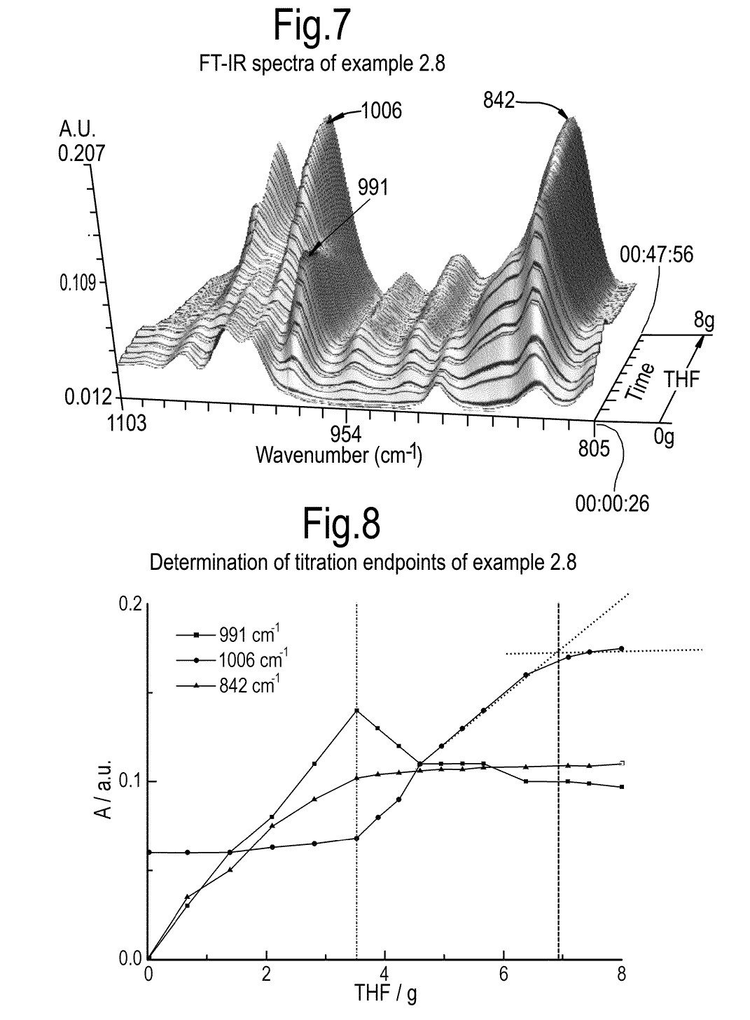

The procedure of example 2.7 was repeated with 20.003 g of IL-1 using THF (supplied by Aladdin Company) as titrant instead of acetone. FIG. 7 shows that during titration the absorption peaks at 991 cm⁻¹, 842 cm⁻¹ and 1006 cm⁻¹ appeared, and the intensity of these three peaks increased when THF was added in portions. The titration end points were determined at the point when the intensity of the peaks at 991 cm⁻¹ and 842 cm⁻¹ reached maxima, and/or the peak at 1006 cm⁻¹ just appeared sharply (FIG. 8). The THF usage was 3.527 g at this titration end point. A second titration end point using twice the amount of THF was found when the peak at 1006 cm⁻¹ reached its maximum. The “activity index” of IL-1 ionic liquid was 0.245 mol indicator/100 g of IL.

### Example 2.9 Determination of Catalytic Activity of IL-1 with Infrared Spectroscopy by Titration of IL-1 with Ethanol and Using Dichloromethane (DCM) as Solvent

IL-1 (20.005) g was placed in a 100 mL flask under N₂ atmosphere and 15 mL of DCM (supplied by Aladdin Company) dried over mol sieves was added. The mixture was stirred continuously during the titration. The titration was performed by addition of ethanol (supplied by Aladdin Company) in portions while FT-IR spectra of the mixture were recorded in situ by an infrared detection apparatus at equal time intervals.

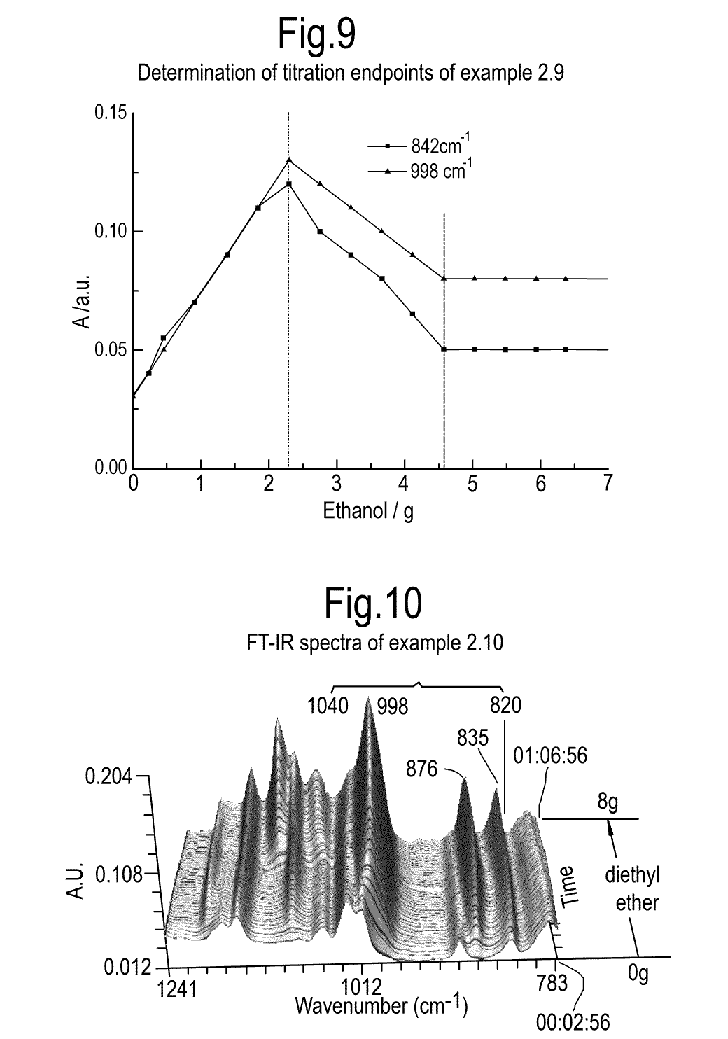

Absorption peaks at 998 cm⁻¹ and 842 cm⁻¹ appeared and their intensities increased gradually when ethanol was added in portions. The intensity changes of these two peaks were tracked by the in-situ infrared apparatus and plotted against the amount of ethanol added (FIG. 9). The titration end point was determined when the intensity of the peaks at 998 cm⁻¹ and 842 cm⁻¹ reached maxima (FIG. 9). The ethanol usage was 2.298 g at this titration end point. A second titration end point using twice the amount of ethanol was found when peaks at 998 cm⁻¹ and 842 cm⁻¹ had decreased to a stable level; this amount of ethanol needs to be divided by 2, to be used in the calculation of the catalytic activity. The catalytic activity of IL-1 ionic liquid defined as “activity index” was 0.249 mol indicator/100 g of IL.

### Example 2.10 Determination of Catalytic Activity of IL-1 with Infrared Spectroscopy by Titration of IL-1 with Diethyl Ether

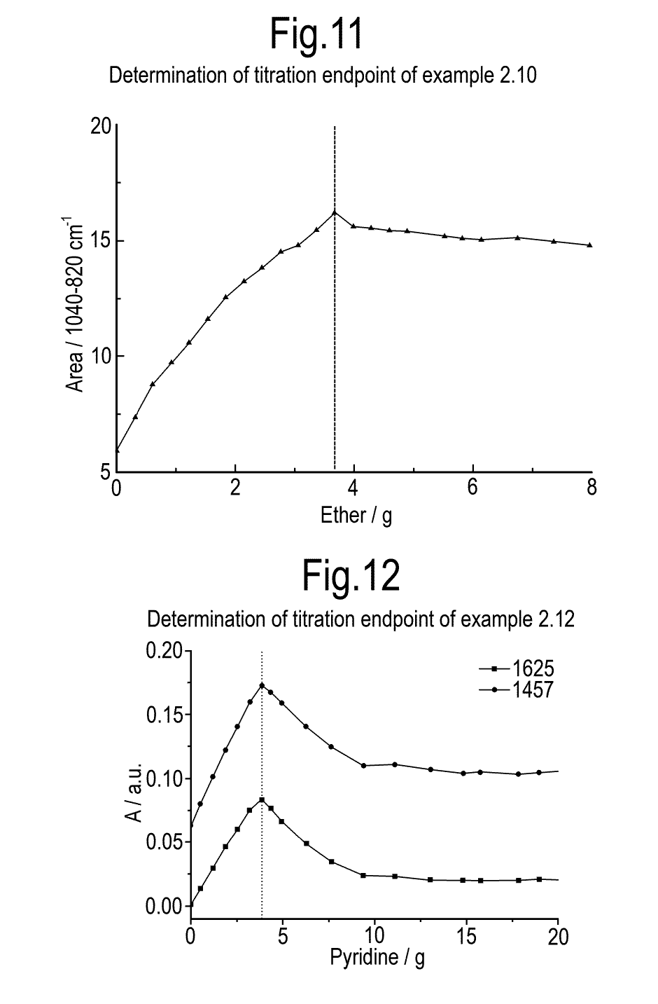

The procedure of example 2.9 was repeated with 20.001 g of IL-1, 16 mL of DCM and using diethyl ether (supplied by Aladdin Company) as titrant instead of ethanol. FIG. 10 shows that during titration absorption peaks at 998 cm⁻¹, 876 cm⁻¹ and 835 cm⁻¹ appeared, and the intensity of these peaks increased gradually when diethyl ether was added in portions. The titration end point was determined when the total integral area of the peaks in the range of 820 cm⁻¹ to 1040 cm⁻¹ reached maximum (FIGS. 10 and 11). The diethyl ether usage was 3.675 g at the titration end point. The “activity index” of IL-1 ionic liquid was 0.248 mol indicator/100 g of IL.

### Example 2.11 Determination of Catalytic Activity of IL-6 with Infrared Spectroscopy by Titration of IL-6 with Nitrobenzene

The procedure of example 2.1 was repeated determining the activity of IL-6 (20.001 g) instead of IL-1. The nitrobenzene usage was 4.948 g at the titration end point. The “activity index” of IL-6 ionic liquid was 0.201 mol indicator/100 g of IL.

### Example 2.12 Determination of Catalytic Activity of IL-1 with Infrared Spectroscopy by Titration of IL-1 with Pyridine and Using Dichloromethane (DCM) as Solvent

The procedure of example 2.9 was repeated with 20.001 g of IL-1, 16 mL of DCM and using pyridine (supplied by Aladdin Company) as titrant instead of ethanol. Absorption peaks at 1625 cm⁻¹ and 1457 cm⁻¹ appeared and their intensities increased gradually when pyridine was added in portions. The intensity changes of these two peaks were tracked by the in-situ infrared apparatus and plotted against the amount of pyridine added (FIG. 12).

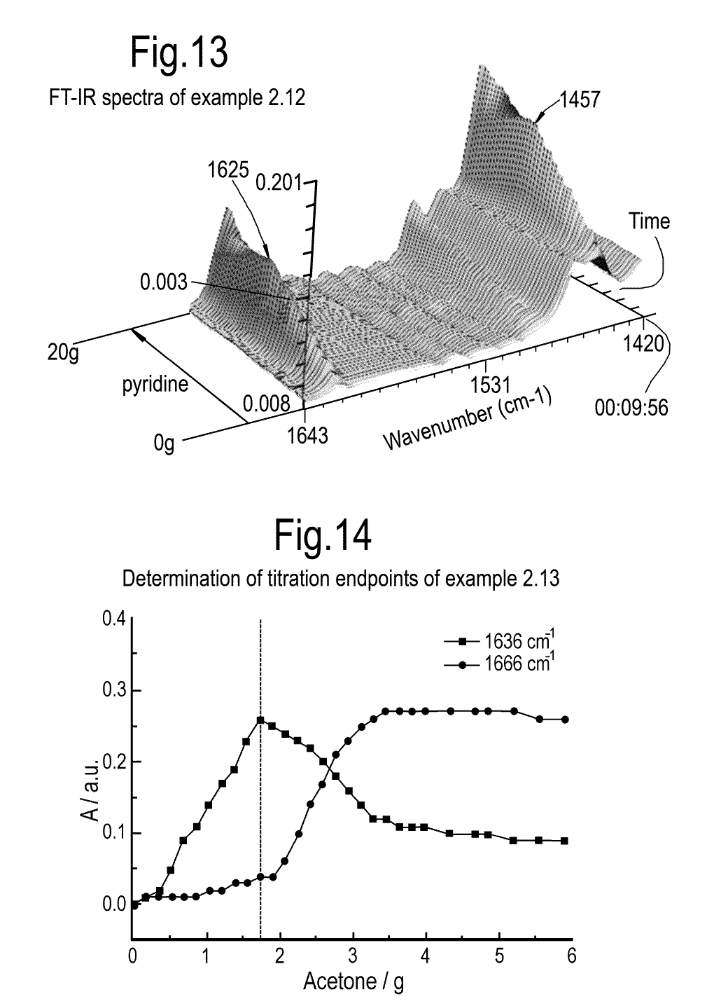

The titration end point was determined when the intensity of the peaks at 1625 cm⁻¹ and 1457 cm⁻¹ reached maxima (FIG. 13). The pyridine usage was 3.835 g at this titration end point. The “activity index” of IL-1 ionic liquid was 0.243 mol indicator/100 g of IL.

### Example 2.13 Determination of Catalytic Activity of IL-7 with Infrared Spectroscopy by Titration of IL-7 with Acetone and Using Dichloromethane (DCM) as Solvent

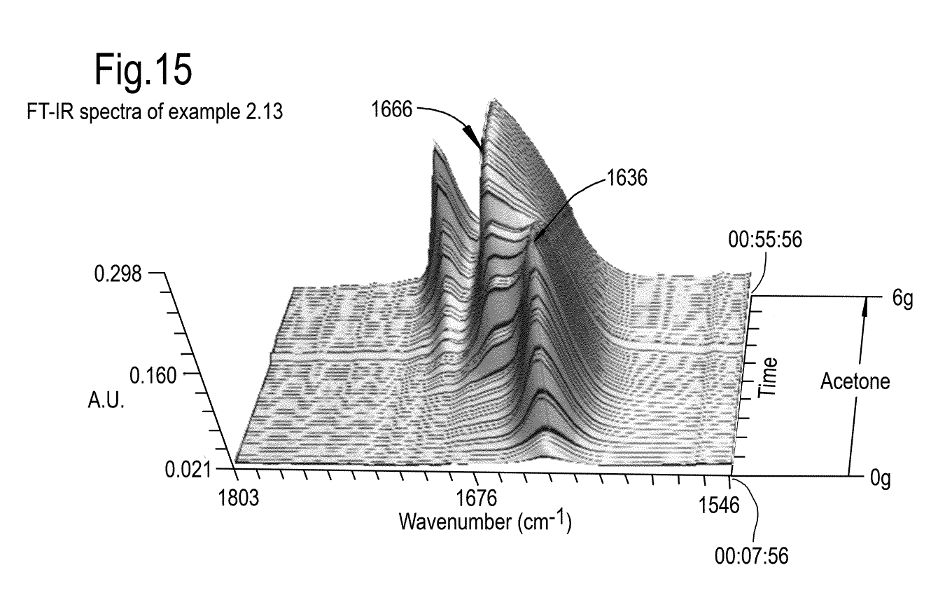

IL-7 (20.003) g was placed in a 50 mL flask under N₂ atmosphere and was stirred continuously during the titration. The titration was performed by addition of acetone (supplied by Aladdin Company) in portions while FT-IR spectra of the mixture were recorded in situ by an infrared detection apparatus at equal time intervals. FIG. 14 shows that during titration initially an absorption peak at 1666 cm⁻¹ appeared and when it reached its maximum another peak at 1636 cm⁻¹ appeared, while acetone was added in portions. The intensity changes of these two peaks were tracked by the in-situ infrared apparatus and plotted against the amount of acetone added (FIG. 15). The titration end points were determined at the moment that the intensity of the 1636 cm⁻¹ peak reached its maximum and when the intensity of the 1666 cm⁻¹ was not increasing anymore upon the addition of acetone. The acetone usage was 1.728 g at the first titration end point (and 3.4 g at the second titration end point). The “activity index” of IL-7 ionic liquid was 0.149 mol indicator/100 g of IL.

TABLE 1
Catalytic activity of IL defined as “activity
index” as determined with infrared spectroscopy in
examples 2.1-2.12

| | | | “Activity index” of IL |
|---|---|---|---|
| | | | (mol organic |
| example | Titre | Indicator | compound/100 g IL) |
| 2.1 | IL-1 | nitrobenzene | 0.245 |
| 2.2 | nitrobenzene | IL-1 | 0.243 |
| 2.7 | IL-1 | acetone | 0.246 |
| 2.8 | IL-1 | THF | 0.245 |
| 2.9 | IL-1 | ethanol | 0.249 |
| 2.10 | IL-1 | diethyl ether | 0.248 |
| 2.12 | IL-1 | pyridine | 0.243 |
| 2.3 | IL-2 | nitrobenzene | 0.209 |
| 2.4 | IL-3 | nitrobenzene | 0.169 |
| 2.5 | IL-4 | nitrobenzene | 0.123 |
| 2.6 | IL-5 | nitrobenzene | 0.068 |
| 2.11 | IL-6 | nitrobenzene | 0.201 |

### Example 3.1 Alkylation Tests with IL-6

350 g of composite IL-6 was placed into a 1000 mL autoclave. The autoclave was closed, the stirrer was started, and the temperature inside the autoclave was controlled at 20° C. C4 feed with an I/O ratio (isobutane/2-butene) of 10:1 (mol/mol) was pumped through a filter and a dryer, and then entered into the autoclave. The feed rate was controlled at 900 mL/h by the plunger pump. The pressure in the autoclave was maintained at 0.5 MPa to keep the reactants and product in liquid phase. During reaction and filling the autoclave, the reaction system was separating into two phases due to the differences in density. The upper part of the reaction mixture in the autoclave was the unreacted feed and products, while the lower part consisted of a mixture of composite ionic liquid and hydrocarbons. The upper part of the reaction mixture was collected via an overflow into a collection tank. Samples were taken from the overflow after certain amounts of feed fed into the autoclave to check for the conversion of 2-butene. After certain amounts of feed fed into the autoclave the feed and the stirrer were stopped and after 5 min a sample of the lower part, consisting mainly of composite ionic liquid, was taken from the bottom of the autoclave; at the same moment also a sample was taken from the overflow to check for the conversion of 2-butene (see Table 2), after which the stirring and the C4 feed was continued. The samples taken from the bottom of the autoclave were decompressed to remove dissolved hydrocarbon and were subsequently centrifuged to remove solid formed during reaction. The procedure of example 2.7 was used to determine the catalytic activity of composite ionic liquid obtained from the samples taken from the bottom of the autoclave (see Table 2).

TABLE 2
Catalyst activity of CIL measured as “activity
index” and butene conversion along with alkylation
process in example 3.1

| C4 feed | | | Acetone | |
|---|---|---|---|---|
| fed to | Olefin | CIL sample | titrant usage | “activity index” |
| autoclave | conversion | size* | at end point | (mol acetone/ |
| (kg) | (%) | (g) | (g) | 100 g CIL) |
| 0 | — | 2.14 | 0.25 | 0.201 |
| 8.5 | 100 | 2.26 | 0.25 | 0.190 |
| 15.0 | 100 | 2.28 | 0.17 | 0.128 |
| 27.0 | 100 | 2.31 | 0.09 | 0.067 |
| 39.0 | 100 | 2.30 | 0.07 | 0.052 |
| 42.5 | 100 | 2.34 | 0.03 | 0.022 |
| 44.7** | 67 | 2.31 | | |
| 45.0 | 25 | 2.33 | 0.02 | 0.015 |

*Sample size after decompression and solids removal
**only sample of overflow was taken.

### Example 3.2 Alkylation Tests with IL-7

The procedure of example 2.8 was repeated with 300 g of composite IL-7. After 25.7 kg of C4 feed (I/O ratio: 10:1 (mol/mol)) fed into the autoclave, the conversion of 2-butene was lower than 90% (81%). Then the feed and the stirrer were stopped and after 30 minutes a sample of the lower part, consisting mainly of ionic liquid, was taken from the bottom of the autoclave (IL-7-deactivated). At the same moment also a sample was taken from the overflow to check for the conversion of 2-butene (48%). The procedure of example 2.7 was used to determine the catalytic activity of composite ionic liquid IL 7 obtained from the samples taken from the bottom of the autoclave (see Table 3).

TABLE 3
Catalyst activity of CIL measured as “activity
index” and butene conversion along with alkylation
process in example 3.2

| C4 feed | | | |
|---|---|---|---|
| fed to | Olefin | CIL sample | “activity index” |
| autoclave | conversion | size* | (mol acetone/ |
| (kg) | (%) | (g) | 100 g CIL) |
| 0 | — | 2.14 | 0.150 |
| 20 | 100 | 2.26 | 0.047 |
| 25.7* | 81 | 2.28 | n.d |
| 26.0 | 48 | 2.31 | 0.011 |

*only sample of overflow was taken.

### DISCUSSION

The “activity indices” as determined in examples 2.1-2.12 are summarized in Table 1 showing that with different organic compounds used as indicators similar activities are determined (within the error of the experiment, see examples 2.1, 2.2, 2.7-2.10 and 2.12). The “activity index” is different for each type of ionic liquid.

Further, Tables 1 and 2 show that the activity index of the ionic liquids decreased while the amount of AlCl₃ and AlBr₃ in the ionic liquids decreased. This indicates that a high amount of Lewis acidity, determined by the amount of AlCl₃ and AlBr₃, may influence the catalytic activity (activity index) in a positive manner.

Example 3.1 and 3.2 show that the activity index can be monitored by sampling ionic liquid from the continuous alkylation process. The results in Table 2 and 3 show the activity index, being a measure of the Lewis acidity, decreased gradually. This indicates that deactivated ionic liquid has little, but insufficient Lewis activity to completely convert the olefin in the alkylation reaction. By using the method according to the present invention it can be determined at which activity index the alkylation activity is too low for total conversion of the olefin.

1. A process for monitoring the catalytic activity of an ionic liquid, comprising the steps of:
(a) providing an acidic ionic liquid;
(b) providing an organic compound which contains a nitrogen group, oxygen group and/or sulphur group;
(c) adding a portion of the organic compound to a sample of the ionic liquid or adding a portion of the ionic liquid to a sample of the organic compound;
(d) recording an infrared spectrum of a mixture as obtained in step (c) to obtain at least one absorption peak;
(e) repeating steps (c) and (d) until at least one absorption peak obtained in step (d) reaches a maximum value or a minimum value;
(f) determining at the maximum value or minimum value of the absorption peak of step (e): the total amount of the organic compound added in portions to the sample of the ionic liquid or determining the total amount of the ionic liquid added in portions to the sample of organic compound;
(g) calculating the catalytic activity of the ionic liquid on the basis of: the total amount of the organic compound added in portions as determined in step (f) or the total amount of ionic liquid added in portions as determined in step (f).

2. The process according to claim 1, wherein the organic compound which contains a nitrogen group, oxygen group and/or sulphur group is selected from the group consisting of alcohols, ketones, ethers, tetrahydrofurans, aldehydes, mercaptans, sulphur ethers, thiophenes, pyridines, nitro-aromates and derivatives thereof.

3. The process according to claim 1, wherein the organic compound which contains a nitrogen group, oxygen group and/or sulphur group is selected from the group consisting of ethanol, acetone, diethyl ether, tetrahydrofuran, nitrobenzene, meta-methyl nitrobenzene, pyridine and 2,6-dimethyl pyridine.

4. The process according to 3, wherein the organic compound or the ionic liquid is used as a mixture using a solvent as diluent.

5. The process according to, wherein the infrared spectrum of steps (d) and (e) is recorded in situ during step (c), (d) and (e).

6. The process according to claim 1, wherein in step (d) one or more absorption peaks are obtained corresponding to one or more products between the ionic liquid and the organic compound.

7. The process according to claim 6, wherein in step (e) at least one absorption peak corresponding to a product between the ionic liquid and the organic compound reaches a maximum.

8. The process according to, wherein in step (c) a portion of the organic compound is added to a sample of ionic liquid.

9. The process according to claim 8, wherein in step (e) a first absorption peak corresponding to a first product between the ionic liquid and the organic compound reaches a maximum and at further repeating steps (c) and (d) a second absorption peak corresponding to a second product between the ionic liquid and the organic compound reaches a maximum.

10. The process according to claim 8, wherein in step (e) at least one absorption peak corresponding to a product between the ionic liquid and the organic compound reaches a maximum and at further repeating steps (c) and (d) the same absorption peak reaches a minimum.

11. The process according to claim 7, wherein in step (f) the total amounts of the organic compound added in portions to the sample of the ionic liquid is determined at which in step (e) one or more absorption peaks corresponding to a product between the ionic liquid and the organic compound reach a maximum or a minimum after first having reached a maximum.

12. The process according to claim 1, wherein in step (c) a portion of the ionic liquid is added to a sample of the organic compound.

13. The process according to claim 12, wherein in step (d) at least one absorption peak is obtained corresponding to the organic compound.

14. The process according to claim 12, wherein in step (e) the absorption peak corresponding to the organic compound reaches a minimum.

15. The process according to claim 1, wherein in step (f) the total amount of the ionic liquid added in portions to the sample of organic compound is determined at which in step (e) a minimum is reached of the absorption peak corresponding to the organic compound or a maximum of the absorption peak corresponding to the product between the ionic liquid and the organic compound.

16. The process according to claim 1, wherein in step (g) the catalytic activity of the ionic liquid is determined by the ratio of the total amount of the organic compound added in portions as determined in step (f) and the amount of the sample of ionic liquid of step (c).

17. The process according to claim 1, wherein in step (g) the catalytic activity of the ionic liquid is determined by the ratio of the total amount of the sample of organic compound of step (c) and the total amount of ionic liquid added in portions as determined in step (f).

18. The process to prepare an alkylate product, the process at least comprising the steps:
(aa) providing a hydrocarbon mixture comprising at least an isoparaffin or an aromatic hydrocarbon and an olefin;
(bb) subjecting the mixture of step (aa) to an alkylation reaction between the isoparaffin or the aromatic hydrocarbon and the olefin, wherein the hydrocarbon mixture is reacted with an ionic liquid to obtain an effluent comprising at least an alkylate product;
(cc) separating the effluent of step (bb), thereby obtaining a hydrocarbon-rich phase and an ionic liquid-rich phase;
(dd) fractionating the hydrocarbon-rich phase of step (cc), thereby obtaining at least the alkylate product and an isoparaffin-comprising stream or an aromatic hydrocarbon-comprising stream; and
(ee) recycling of the ionic liquid-rich phase of step (cc) to step (bb), wherein the catalytic activity of the ionic liquid of step (bb) and of the ionic liquid rich phase of step (cc) is determined according to claim 1.
